(19) United States
(12) Patent Application Publication
(10) Pub. No.: US 2026/0336367 A1
(43) Pub. Date: Oct. 29, 2026

(54) CONTROLLED ENVIRONMENT AGRICULTURE SYSTEM

(71) Applicant: TOMPHYZX.LLC, Dundee, NY (US)

(72) Inventor: Thomas Dunbar, Dundee, NY (US)

(21) Appl. No.: 19/111,577

(22) PCT Filed: Sep. 13, 2023
(86) PCT No.: PCT/US2023/032630

§ 371 Date: Mar. 13, 2025

Related U.S. Application Data
(60) Provisional application No. 63/406,102, filed on Sep. 13, 2022.
(60) Provisional application No. 63/453,271, filed on Mar. 20, 2023.

(51) Int. Cl. B60H 3/00; A01G 9/24; A61L 9/20; B60H 1/34

(52) U.S. Cl. CPC B60H 3/0078; A01G 9/246; A01G 9/249; A61L 9/20; B60H 1/3407

(57) ABSTRACT

An illumination and pathogen reduction system for controlled environment agriculture is provided having an airflow pathogen reduction system having a first flow path and a UV-C source optically coupled to the first flow path for treating a volume of air, the volume of treated air expelled along a second flow path, an illumination source having photosynthetically active radiation directed towards an illumination field, and a turbulence generator within the second flow path, wherein the turbulence generator provides a turbulent flow of treated air towards a desired location.

### CROSS-REFERENCE TO RELATED APPLICATIONS

The present application claims the benefit of U.S. provisional patent application 63/406,102 filed Sep. 13, 2022, entitled ILLUMINATION AND PATHOGEN REDUCTION SYSTEM, METHOD AND DEVICE FOR CONTROLLED ENVIRONMENT AGRICULTURE and U.S. provisional patent application 63/453,271 filed Mar. 20, 2023 entitled ILLUMINATION AND PATHOGEN REDUCTION SYSTEM, METHOD AND DEVICE FOR CONTROLLED ENVIRONMENT AGRICULTURE, the entire disclosures of which are hereby incorporated by reference.

### BACKGROUND OF THE INVENTION

### Field of the Invention

The present disclosure is related to controlled environment agricultural systems and more particularly, to a controlled environment agricultural system having an airflow system, an illumination source and a first air deflector. The present disclosure is further related to an illumination and pathogen reduction system for a controlled environment agricultural system having a clean airflow system, including an air duct system that generates a turbulent air movement through the growth canopy.

### Description of Related Art

Controlled Environment Agriculture (CEA) systems are designed to provide the optimal growing conditions for plants throughout their growth cycle from nursery to harvest. Unfortunately, these conditions are often optimal for the propagation and growth of micro-organisms such as viruses, molds, fungi, and bacteria that prey on the host plants. The emerging cannabis industry specifically has powdery mildew, gray mold, and Aspergillus potentials that can cause health issues from inhalation or ingesting active spores from these pathogens.

Regulations for commercial distribution requires testing for these pathogens and has been implemented in many states. Positive tests have become a significant yield loss for CEA growers. Further concerns for workers health exist where outbreaks may occur in closed HVAC system grow rooms. In a closed system, outside air is not brought in or inside air exhausted. This requires an additional effort to ensure the air is being properly treated through control mechanisms such as filtration removal and germicidal inactivation. Airborne spores are released from active pathogen sites within the plant foliage or soil distribution or are brought in by workers. In addition, closed systems increase the probabilities that co-workers may transmit diseases from airborne pathogens such as coronavirus and influenza.

Examples of a method and system for UV germicidal inactivation of pathogens by RNA-DNA damage to a level that overcomes pathogen repair mechanisms and greatly reduces the ability to replicate subsequently causing pathogen death are disclosed in U.S. Pat. No. 11,357,882, which is hereby incorporated by reference.

### BRIEF SUMMARY OF THE INVENTION

Embodiments of the present disclosure provide a botanical illumination, germicidal illumination, and air circulation design pathogen reduction system and method.

According to one aspect of the present disclosure, there is provided an illumination and pathogen reduction system for controlled environment agriculture comprising an airflow pathogen reduction system having a first flow path and a UV-C source optically coupled to the first flow path for treating a volume of air, the volume of treated air expelled along a second flow path, an illumination source having photosynthetically active radiation directed towards an illumination field, and a turbulence generator within the second flow path, wherein the turbulence generator provides a turbulent flow of treated air towards a desired location.

In another aspect of the present disclosure, the illumination and pathogen reduction system includes an HVAC system having a controller, an air handling unit, a supply duct line, and a return duct line, a return flow control box fluidly connected to the return duct line of the HVAC system, and at least one airflow pathogen reduction system, the airflow pathogen reduction system having an airstream intake for receiving a volume of untreated air located at a first position, an airstream duct for moving a volume of treated air to a room, an input power source operably connected to the UV-C source, an illumination channel along the first flow path having an inner highly UV-C reflective surface providing an optical effective power that exceeds the input power to the UV-C source, wherein the air handling unit of the HVAC system is fluidly connected to the airstream intake, the air handling unit configured to impart a flow from the airstream intake to the airstream duct, and an airflow control system fluidly connected to the airstream duct.

Another aspect of the present invention includes a method of treating a living organism in a controlled environment agricultural system comprising generating treated air through an airflow pathogen reduction system having a UV-C source for treating a volume of air along a first flow path, expelling the treated air towards a turbulence generator, the turbulence generator configured to provide turbulent air in a turbulent air zone, exposing a living organism to the turbulent air in the turbulent air zone to provide a worked airflow, and collecting the worked airflow having contaminants from the living organism in a return duct.

Yet another aspect of the present invention includes an indoor grow room enclosure comprising an airflow pathogen reduction system having at least one illumination chamber subassembly comprising first flow path and a UV-C source optically coupled to the first flow path for treating a volume of air, the volume of treated air expelled along a second flow path, a turbulence generator within the second flow path, the turbulence generator providing turbulent airflow in a turbulent zone of treated air, an illumination source directed towards an illumination zone, the illumination source capable of illuminating the illumination zone, a first base having a support surface for receiving an object requiring illumination, the base having an adjustable height for positioning the object within the illumination zone and the turbulent zone of treated air, wherein the turbulent airflow is passed through the object providing worked airflow having contaminants, and a return duct for removing the worked airflow from an indoor grow room enclosure.

### BRIEF DESCRIPTION OF THE SEVERAL VIEWS OF THE DRAWINGS

FIG. 1 is a perspective view of an exemplary embodiment of an illumination and illumination and pathogen reduction system.

FIG. 2 is a partial front view of an exemplary embodiment of the illumination and airflow pathogen reduction system.

FIG. 3 is a perspective view of a portion of an exemplary embodiment of an lighting airfoil housing.

FIG. 4 is a perspective view of a portion of an exemplary embodiment of another lighting airfoil housing.

FIG. 5A is a front view of an exemplary embodiment of a section of a supply duct showing an exit port.

FIG. 5B is a front view of another exemplary embodiment of a section of a supply duct showing an exit port.

FIG. 6 is a front view of an exemplary embodiment of an electrical conduit.

FIG. 7 is a partial cross section view of an exemplary embodiment of the illumination and airflow pathogen reduction system of FIG. 1 illustrating the center row.

FIG. 8 is a perspective view of an exemplary embodiment of an outside of a grow room showing a return duct.

FIG. 9 is a perspective view of an airflow pathogen reduction system suitable for use in practicing exemplary embodiments of this disclosure.

FIG. 10 is a side section view of a portion of the airflow pathogen reduction system of FIG. 1 suitable for use in practicing exemplary embodiments of this disclosure.

FIG. 11 is a schematic drawing of an exemplary embodiment of the airflow pathogen reduction system suitable for use in practicing exemplary embodiments of this disclosure.

FIG. 12 is a schematic drawing of an exemplary embodiment of the airflow pathogen reduction system suitable for use in practicing exemplary embodiments of this disclosure.

FIG. 13 is a representative drawing of an exemplary embodiment of a grow room area.

FIG. 14 is a schematic drawing of an exemplary embodiment of the airflow pathogen reduction system for a plurality of grow rooms suitable for use in practicing exemplary embodiments of this disclosure.

FIG. 15 is a perspective view of an exemplary embodiment of an illumination and airflow pathogen reduction system for an enclosure.

FIG. 16 is a partial cross section view of an exemplary embodiment of an illumination and airflow pathogen reduction system for an enclosure of FIG. 15.

FIG. 17 is a schematic drawing of an exemplary embodiment of the illumination and pathogen reduction system for an enclosure suitable for use in practicing exemplary embodiments of this disclosure.

FIG. 18 is a schematic drawing of an exemplary embodiment of the illumination and pathogen reduction system for a vertical farming grow room suitable for use in practicing exemplary embodiments of this disclosure.

FIG. 19 is a logic diagram showing a method of an exemplary embodiment of this disclosure.

### DETAILED DESCRIPTION OF THE INVENTION

It should be appreciated that the same reference numbers appearing in different figures identify the same structural elements of the present invention. While the description of the present invention includes what is currently considered to be the preferred configurations, it should be appreciated that the present invention is not limited to such configurations. Moreover, it should be appreciated that the present invention is not limited to the particular methodology, materials and modifications described herein and that the terminology used herein is not intended to limit the scope of the present invention. The scope of the present invention is therefore to be determined solely by the appended claims.

The present disclosure contemplates that many changes and modifications may be made. Therefore, while the presently-preferred form of the apparatus and method has been shown and described, and several modifications and alternatives discussed, persons skilled in the art will readily appreciate that various additional changes and modifications may be made without departing form the scope of the invention, as defined and differentiated by the following claims.

A controlled environment agriculture (CEA) system is a technology based approach to growing plants in a controlled environment. CEA allows certain environmental parameters to be controlled, including lighting, temperature, humidity, nutrient levels, carbon dioxide concentrations and oxygen concentrations. CEA provides high-value crops at a high yield, which is efficient and environmentally friendly. CEA offers many advantages, including growing crops year-round, reducing crop damage from pollution, pests, drought, flooding, and harsh weather, and using less water and land. Providing optimal growing conditions for plants, however, also creates an environment for pathogens to thrive. Embodiments of the disclosure can provide an optimal growing environment while reducing pathogens in the system.

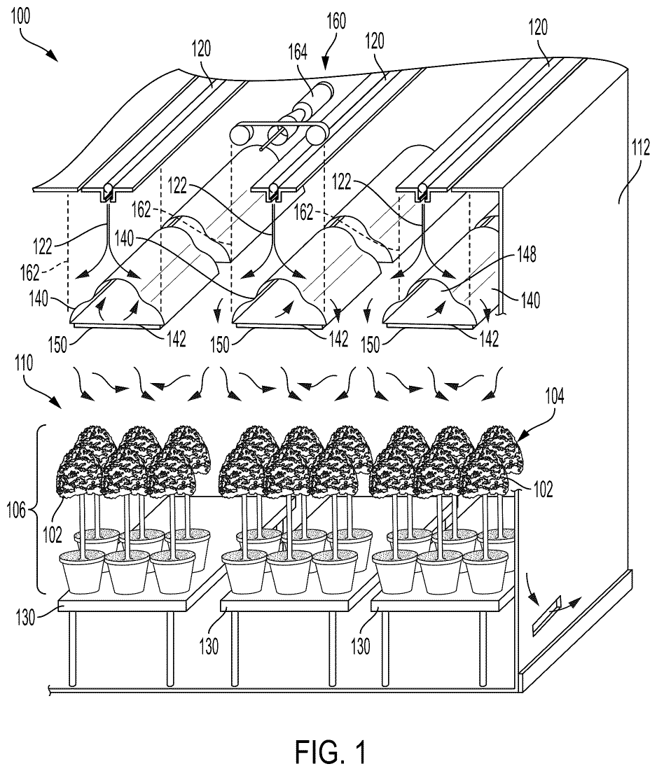

Referring to FIG. 1, a portion of an illumination and airflow pathogen reduction system 100 is shown in an interior 110 of a grow room 112 where various living organisms in the kingdoms Protista, Fungi, and Plantae 102 can be grown. That is, living organisms include plants, algae and fungi, but not living organisms in the Animalia kingdom. The illumination and pathogen reduction system 100 may include at least one HVAC unit, fans and associated air handling equipment and ducts. The illumination and pathogen reduction system 100 may further include sensors for detecting and controlling at least one environmental condition, including air temperature, relative air humidity, airflow speed, oxygen and carbon dioxide concentration. The illumination and pathogen reduction system 100 includes at least one supply duct 120. In an embodiment, each supply duct 120 is positioned substantially parallel to the axis of a crop table 130 array. As shown in FIG. 1, in an embodiment, a turbulence generator 140 is spaced from the supply duct 120. In an embodiment, the turbulence generator 140 produces turbulent air within a turbulent zone 124. In certain embodiments, the turbulence generator 140 also directs treated air towards a desired location. The turbulence generator 140 in one embodiment is within a lighting housing 142, which includes an illumination source 150 configured to illuminate an illumination field. It should be appreciated that the illumination source 150 can be selected according to optimize various factors including crop type, growth stage, specific growth goals (e.g., enhancing flavor, speeding up growth, increasing yields, etc.) and the cost of the illumination source. An example of an illumination source 150 that can be used is a light emitting diode (LED) light source, such as an LED illumination panel. LEDs are energy efficient, have a desirable lifespan, and the ability to produce specific light spectra. Moreover, LEDs can be tailored to emit blue, red, white, far-red, or even ultraviolet (UV) and infrared (IR) light. Thus, LEDs are particularly adaptable to various stages of plant growth and for specific crop needs. LEDs also generate less heat than High Pressure Sodium (HPS) lamps or Metal Halide (MH) lamps, which can be advantageous in a controlled environment. It should be appreciated, however, that other illumination sources can be used. High-Intensity Discharge (HID) lamps or fluorescent lights are examples of illumination sources that can be used. T5 grow lights are high-output fluorescent lights that are frequently selected for seed starting and early vegetative growth as a result peaks in the blue spectrum. Further examples of illumination sources include Ceramic Metal Halide (CMH) or Light Emitting Ceramic (LEC) lamps, which are types of MH lights using ceramic as part of the bulb. This may improve efficiency and the spectral output. Plasma lighting is another possible illumination source. Plasma lighting has a spectrum similar to sunlight and is energy efficient. Additional examples include HPS lamps which produce a red-orange light, which is beneficial for flowering and fruiting plants. They are highly energy-efficient. An MH lamp is another example of an illumination source. MH lamps produce a blue-white light that is ideal for vegetative growth and are especially useful in the early stages of plant growth. Other illumination sources may include incandescent and halogen bulbs, compact fluorescent lamps (CFLs), vertical farming specific LEDs, green light, and supplemental lighting systems. Supplemental lighting systems include UV and IR lighting solutions, which can stimulate specific plant responses, such as improved flavor, color or pest resistance. Although plants predominantly use red and blue light for photosynthesis, it has been found that green light can penetrate deeper in to plant canopies and influence certain physiological processes. Thus, some CEA illumination sources include green light in the illumination spectrum.

The illumination source 150, such as LED illumination, provides a finely tuned spectra optimal for plant, algae or fungi 102 growth and is adjustable throughout the growth cycle. High efficiency illumination sources 150, such the LED light source, aid to control overall temperature variations within a grow volume 104 of each plant 102. By “grow volume” it is meant a volume of vegetative or other living organism material located a distance from the illumination source 150.

Photosynthetically active radiation (PAR) is a term generally used in association with the light output of agricultural illumination systems. It defines the types of light required to support photosynthesis in plants and algae since plants convert light energy into chemical energy. More specifically, PAR encompasses the visible light spectrum between 400 nm and 700 nm, which includes both blue (400-500 nm) and red (600-700 nm) light. It should be appreciated that PAR values (typically measured in micromoles/m²/s) and duration of exposure (daily light integral (DLI)) can be selected to influence plant growth, flowering, fruiting and the general health of the plant. That is, by determining an optical light quality and quantity, growth, yield and other desirable traits can be maximized. Photosynthetic photon flux (PPF) is the total light (photons) emitted per second by a light source with a wavelength of 400-700 nm. The number of micro moles of photons emitted by an artificial light source per second is measured in micro moles per second and expressed in micromol/s. PPF and PPF density correspond to the number of micro moles radiated by the light source per square meter per second. The unit is micromol/m2s. Photosynthetic photon flux surface density (PPFD) is a surface measurement of the light (photons) reaching the target per second.

The optimal wavelength spectrum can vary for different stages of growth of plants and algae. Since LEDs can be engineered to emit specific wavelengths, for example, blue, red, white, far-red, or even ultraviolet (UV) and infrared (IR) light, LEDs are often selected as the illumination source 150. Full spectrum LEDs include the entire PAR range and may include parts of the spectrum outside of the PAR range to mimic natural sunlight. In some configurations, growers can select spectral ranges to influence plant growth. For example, different spectrum ranges may be selected at different times in the grow cycle. More specifically, a grower may select the near ultraviolet spectrum range (300 nm to 400 nm) during a plant's flowering stages to induce a desired plant response. In some cases, using UV to cause stress in the plant can be beneficial for the plant. For example, cannabis produces “cannabinoids as sunscreen.” Using far red 700-850 nm causes a reaction in the plant that is in a shadow and triggers faster growth. Thus, in some embodiments, an array of LEDs with junctions designed for different peak wavelengths can be used to enable “tuning” of the spectrum for each stage of plant maturity. In some configurations, the tunable range is between 300 nm and 850 nm. Other illumination sources 150 can be selected, each offering certain advantages and disadvantages. For example, HPS lamps provide light in the yellow-orange-red spectrum (570 nm-750 nm) with falls within the PAR range. However, HPS lamps lack significant blue light, which is important for some plant, algae and fungi growth stages or types. On the other hand, MH lamps produce a broader spectrum of light which covers a broader PAR range, including a portion of the spectrum in the blue light range. Alternatively, CMH or LEC provide a balanced spectrum that includes the PAR range, with a more even distribution between blue and red light compared to HPS. Plasma lighting produces a broad-spectrum light that covers the PAR range and is often close to that of natural sunlight. Fluorescent lights produce light across a broad spectrum ranging from approximately 350 nm to 750 nm. As provided above, T5 grow lights are high-output fluorescent lights that generally include the PAR range. T5 grow lights have a blue leaning spectrum and may be desirable for seed starting and early vegetative growth. It should be appreciated that UV lights and IR lights are outside the PAR range, but can still be used to influence plant growth, morphology and secondary metabolite production.

The turbulence generator 140 includes any device that is capable of creating a turbulent flow in a target turbulent zone 124 in a flow path. In an embodiment, the lighting housing 142 includes the illumination source 150 and the turbulence generator 140, wherein the turbulence generator 140 is an air deflector to direct an air discharge from the supply duct 120. While in one embodiment the turbulence generator 140 is shown as a part of the lighting housing 142, it should be appreciated that the turbulence generator 140 may be separate from the lighting housing 142. In one embodiment, the air deflector includes any object, wherein the shape of the object determines the behavior of the airflow. For example, certain shapes cause the airflow to separate from the objects surface early, resulting in a turbulent flow pattern. One example of a possible air deflector includes a deflector body having an undulating outer surface with a leading portion and trailing edges. The turbulent zone is located downstream of the air deflector. Typically, the sides of the turbulence generator 140 are symmetrical to provide generally equal distribution of airflow on each side of the turbulence generator 140. Other air deflectors include bluff bodies, which may include a float or broad rear face, such as a square or rectangular prism, that causes the flow to separate early and creates a downstream turbulent wake. Another type of air deflector includes a cylinder or sphere, which can generate a “von Kármán vortex street” or a repeating pattern of swirling vortices that are shed from alternating sides of the object. Another example includes T-junctions, which are cross-shaped, perpendicular or substantially perpendicular junctions which produce turbulence, especially when the flow rates are high. The air deflector can alternatively be a backward-facing step configuration or a configuration with sharp edges and corners—as flow encounters a sudden increase in height or an abrupt change in direction, flow separation and turbulence can be obtained. Additional air deflectors include perforated plates or cavities. Holes or perforations in a plate can break up flow creating multiple jets and producing turbulence. Similarly, hollow or recessed areas in a surface can cause the flow to recirculate within the cavity, leading to turbulence. Yet another type of air deflector is an object with complex geometries or multiple appendages that cause chaotic flow patterns.

In an embodiment, the lighting housing 142 includes the illumination source 150 and the turbulence generator 140, wherein the turbulence generator 140 is an airfoil designed to create turbulent airflow patterns from air discharge from the supply duct 120. Examples of airfoil devices that can be used include vortex generators, turbulators, stall strips, serrated trailing edges, roughness elements, and slats and slotted flaps. Vortex generators, in some embodiments include small, fin-like devices placed on an upper surface of an airfoil. Vortex generators intentionally generate small vortices, which energize the boundary layer. Turbulators may include thin strips or zigzag patterns placed on the upper surface of glider wings, which trip the laminar flow into becoming turbulent. Stall strips are small triangular devices that attach to the leading edge of an airfoil. Serrated trailing edges are small “teeth” like objects on the trailing edge that break up the vortices form the trailing edge of the airfoil. Roughness elements are purposeful surface roughness or surface imperfections that induce turbulence. Slats and slotted flaps introduce gaps in the airfoil surface. The air from below the airfoil is drawn through the gaps, producing turbulent flow over the top surface of the airflow.

In an embodiment, the airflow discharge from the supply duct 120 is linear, then guided around the outside circumference of the turbulence generator 140, and then recombined beneath the lighting housing 142 in a turbulent manner. An airflow system 100 with a turbulent flow through the grow volume 104 of the plants 102 provides an average downward velocity that matches the desired flow for supply air across the leaves of the plants 102. It should be appreciated that having a plurality of turbulence generators 140 within multiple rows of plants 102 within a grow room 112 can created a cumulative turbulent effect among those rows. That is, flow from one turbulence generator 140 can interfere with the flow from another turbulence generator 140 creating additional turbulence within the grow room 112. Further, turbulent air from an area in a grow room 112 can be directed toward turbulent air flowing from another area in a grow room 112 causing the turbulent air to collide in the turbulent zone, further increasing turbulence.

In one embodiment, air enters the interior 110 of the grow room 112 from at least one linear supply duct 120. Typically, a plurality of supply ducts 120 are positioned along the axis of each crop table 130 and typically the crop tables are arranged in an array. In one embodiment, the grow room 112 includes a plurality of rows of crop tables 130. In an embodiment, the linear supply duct 120 is fluidly connected to an illumination and pathogen reduction system 200 comprising pathogen reduction ducts 202 attached to a supply plenum 290 as described in more detail below. The supply ducts 120 provide treated air to the interior 110 of the grow room 112 toward the plants 102. In FIG. 1, three linear table 130 rows are shown with an array of potted plants 102 on each. Thus, three linear supply ducts 120 are aligned with the length of the crop table array 130. Although three rows of crop tables 130 are shown, it should be appreciated that any number of crop tables 130 can be used.

Air passing along the lighting airfoil housing 142 also serves to transfer heat away from the illumination source 150, for example the LED array set forth in FIG. 1. While more efficient than competing technologies, the power required of the illumination source 150 for optimal plant growth may exceed 500 Watts per lighting housing 142. It should be appreciated, however, that power consumption of LEDs can vary based on the design, purpose, and manufacture of the light. Seedling and propagation lights ranging between about 10 W and 50 W are typically used since seedlings and young plants do not require as much light intensity as mature plants. During the vegetative growth phase of plants such as lettuce, herbs, and other greens, the power consumption of LEDs is typically between about 50 W and 200 W per fixture, although this can vary based on the area covered and light intensity provided. For plants in the flowering and fruiting stage, such as tomatoes, peppers and cannabis, LED lights may be more intense and may cover a broader spectra. Power consumption may range from about 100 W to 600 W or more per fixture. For taller plants, like tomatoes, inter-lighting or intracanopy lights may be selected. These are specialized LED lights placed within the plant canopy which ensure light penetrates deeper into the plant structure. Power consumption for inter-lighting or intracanopy lights may range between about 20 W and 100 W per fixture. Vertical farming LED fixtures are designed for multi-layer cultivation and are generally more compact, with different form factors to maximize space utilization. Typically power consumption is between about 20 W and 150 W per vertical farming LED fixture, depending on crop type and growth stage. Full-spectrum LED fixtures mimic the natural sunlight spectrum and are used for various plant types and growth stages. Power consumption for full-spectrum LED fixtures is typically in the range of about 100 W to 500 W. Supplemental spectra LED fixtures provide specific wavelengths to supplement natural sunlight in greenhouses, such as blue or red LED strips. Power consumption generally ranges from about 10 W to 100 W. Finally, high-efficiency LED fixtures having ultra-high efficiency, using advanced LED chips and drivers to provide maximum light output per watt consumed can be used. These fixtures often deliver more light output at the upper end of their wattage range. It should be understood that power consumption of any LED fixture will also depend on factors based on the manufacturer's design choice, the quality of LED chips used and the efficiency of the drivers and other components. In typical CEA systems, temperature gradients can exist between the floor and the ceiling due to several factors, such as the heat generated by lighting systems, the natural tendency of warm air to rise (convention), equipment operation, and the ventilation system's efficiency. The temperature difference between the floor and the ceiling can impact plant growth, energy consumption and overall system efficiency. Within a CEA system, it is important to monitor temperatures at multiple vertical points to manage the temperature gradient effectively. Examples of temperature gradients within a typical CEA system are provided as follows. In greenhouses, on sunny days, especially in larger, tall greenhouses, the heat from the sun can cause a noticeable temperature gradient. The ceiling or upper canopy can be several degrees warmer than the floor or lower canopy. This is especially pronounced in the absence of adequate ventilation or active cooling. For instance, the temperature near the ceiling might reach 30 °C (86 °F), while the temperature near the floor remains at 25 °C (77 °F), resulting in a 5 °C (9 °F) gradient. In vertical farms, for example, multi-layered vertical farms using LED lighting, the heat from the lights can cause warmer conditions at the top layers compared to the bottom layers, especially if cooling and ventilation are not optimized. For example, the topmost layer could have a temperature of 23 °C (73.4 °F) while the bottommost layer remains at 20 °C (68 °F), a difference of 3 °C (5.4 °F). In indoor grow rooms with high-intensity lights (e.g., HID lamps), significant heat can be generated. If not properly managed, the ceiling can become much warmer than the floor. For example, poorly ventilated rooms may reach temperature differences of 10 °C (18 °F) or more, with ceiling temperatures approaching or exceeding 35 °C (95 °F) and floor temperatures around 25 °C (77 °F). In aquaponics or hydroponics facilities, water temperature can moderate the air temperature. If the water is cooler than the air, it can help reduce temperature gradients. However, if there's a significant amount of warm water (from fish tanks, for instance), it might exacerbate the gradient. The gradient might be subtler, perhaps 2 °C to 4 °C (3.6 °F to 7.2 °F), with the influence of water acting as a thermal buffer. Proper management of the temperature gradient ensures plant health, maximizes growth and improves energy efficiency. Since the air is distributed along the length of the lighting housing 142 in a generally consistent manner along the long axis of the room, a reduction in temperature variations within the grow space 106 and therefore, within the grow volume 104, is obtained. By “grow space” it is meant the area between the table 130 top and the lighting housing 142 where plants 102 are positioned and where treated air and light are directed. To further aid in temperature destratification is the generally downward flow from the warmer top of the grow space 106 to cooler bottom of the grow space 106. The system downward flow balances the temperature gradient by moving and mixing the upper warmer air with the cooler air near the bottom of the grow space. The illumination source 150, for example the LED array, within a lighting airfoil housing 142 provides a uniform heat exchanger as part of the overall system 200 enabling a control algorithm to obtain a balanced temperature distribution from ceiling to floor within the grow room 112. Symmetry and balance control can therefore be achieved with the above described arrangement for CEA.

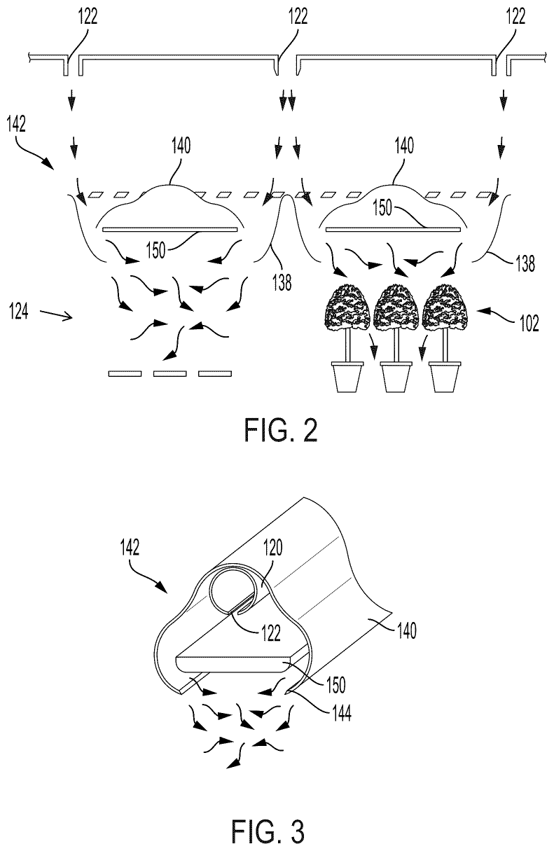

It should be appreciated that the lighting airfoil housing 142 may be individual units above a portion of the table or tables 130 or a continuous lighting airfoil housing 142 along the entire length of the row of crop tables 130. As shown in FIGS. 2 and 3, air movement begins at the supply duct vent 122. In one embodiment, the supply duct vent 122 is in or proximate the ceiling of the grow room 112. As shown in FIG. 3, an alternative and to bring the vent exit force closer to the target turbulent zone 124, the supply duct vent 122 is integrated into the lighting housing 142. The supply duct vent 122 runs linearly in a supply duct 120, which supply duct 120 is inside the lighting airfoil housing 142. Mounting the illumination source 150 into the lighting airfoil housing 142 enables a deflection on a focusing surface 144 directing the air directly into the turbulence zone 124. With symmetry, linear beams of air from each side collide beneath the illumination source 150. In certain embodiments, having the illumination source 150 inside the lighting airfoil housing 142 creates an increased turbulence than can be accomplished with supply vents 122 outside of the lighting airfoil housing 142.

In an embodiment, as shown in FIG. 1, the airflow discharged from the supply duct 120 is generally linearly discharged from the supply vent 122 located above a central peak 148 of the turbulence generator 140, then guided around the outside circumference of the turbulence generator 140, and then recombined beneath the lighting housing 142 in a turbulent manner towards the turbulence zoon 124. Alternatively, as shown in FIG. 2, the supply vent 122 is located off-center from the central peak 148 (but still above the central peak 148). In this embodiment, the lighting airfoil housing 142 may include an additional frame 138 wherein air flows from the vent 122 towards the additional support frame 138 and an outer region of the turbulence generator 140 to direct air in a turbulent manner towards the turbulence zone 124. In one embodiment, the support frame 138 and the lighting housing 142 are coupled such that a height adjustment to the lighting housing 142 includes the support frame 138.

To maintain the optimal lighting distances, the lighting housing 142 can be raised as the plants 102 mature and grow taller. In certain embodiments, a lifting system 160 includes cables 162 connected to at least one motor 164 to raise the lighting housing 142 to meet the desired distance. Since the optimal illumination distance is somewhat constant, the air patterns follow the growth of the plants 102 are constant through the grow cycle. As example, the LEDs are 14 inches above the canopy of the plants 102.

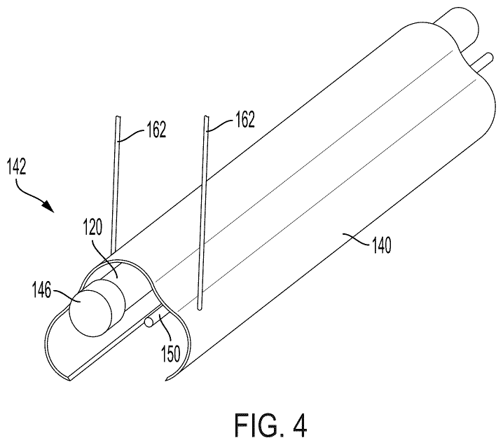

FIG. 4 is a perspective view of a modular LED linear supply air assembly. As shown, a length of standard duct 120 is attached inside the lighting airfoil housing 142. Cables 162 are attached to the lighting airfoil housing 142 to allow a height adjustment. An illumination source 150, such as an LED panel, is mounted inside the lighting airfoil housing 142, and the ends of the supply duct 120 are capped with caps 146 to direct air out through the supply vent 122. The supply duct 120 portions connect in series over the length of the row tables 130. The supply duct 120 portions may include interconnect flanges 134a, 134b, such that the supply duct portions 120 can be connected in series. Thus, both airflow and LED lighting uniformity is maintained along the linear crop tables 130.

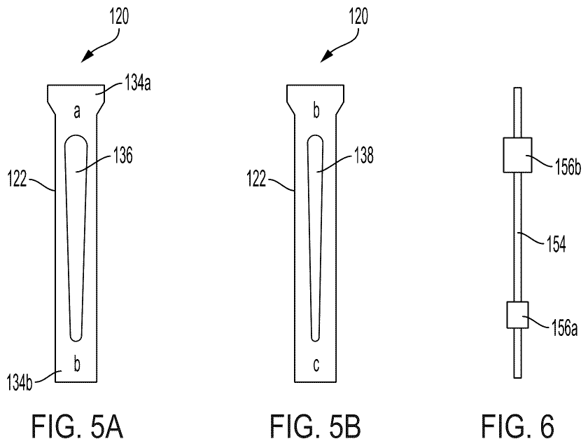

End cap 146 can optionally be two pieces with a fixed section and a removable access panel for installation and maintenance. In one embodiment, the supply vent 122 includes a shaped exit port 136, 138. For example, as shown in FIG. 5A, the exit port 136 may be tapered such that the port end closer to the supply plenum 290 is smaller. In one embodiment, as shown in FIGS. 5A and 5B, multiple supply duct 120 portions are coupled to provide progressively smaller tapers. For example, end “b” of FIG. 5A is coupled to end “b” of FIG. 5B. Thus, the taper of the exit ports 136, 138 are generally progressive smaller as the proximity to the supply plenum 290 is increased (is closer). The tapered slot of the exit ports 136, 138 compensates for lower pressure differential as the distance from the supply plenum 290 increases. This provides a more consistent distribution of air along the supply duct 120. In an embodiment, the cfm per unit length is constant over the length of the coupled supply ducts 120. Given the ductwork system would need to move vertically to keep the illumination source 150 distance and turbulent zone 124 in place with crop growth, some means for the duct 120 to adjust vertically is required. It should be appreciated that flexible ducts and conduits can be used to enable the range of heights desired. In one embodiment, the tapered exit ports 136, 138 have an opening in the range of approximately 0.1″-0.25″ for a supply duct 120 having a length of 5 feet. However, the tapered exit ports 136, 138 may have a range from approximately 0.02″ to 0.25″, increasing in size as the distance from the supply plenum 290 increases.

FIG. 6 shows a possible electrical conduit 154 to be provided within or proximate to the lighting housing 142 having connection boxes shown in two possible alternative positions 156a and 156b, allowing access to wiring. Similar to the supply duct 120 portions, in an embodiment, the electrical conduit connections are provided in a series. In one embodiment, end cap 146 is removable to allow access to the conduit 154 and the connection box 156a or 156b. A turbulent flow of treated air through the grow volume 104 of the plants, algae or fungi 102 increases the plants, algae or fungi 102 exposure to the treated air and reduces the stagnant air zones within and around the plant, algae or fungi 102. It should be appreciated turbulent flow is not laminar. A Reynolds Number is often used to characterize a flow as turbulent. The Reynolds number is the ratio of inertial forces to viscous forces. The Reynolds number is a dimensionless number used to categorize the fluids systems in which the effect of viscosity is important in controlling the velocities or the flow pattern of a fluid. Mathematically, the Reynolds number is defined as

$$Re \cdot = \cdot \frac{uL}{v} = \frac{ρ uL}{μ} \vert ¶$$

where:
ρ is the density of the fluid (SI units: kg/m³)
u is the flow speed (m/s)
L is a characteristic length (m)
μ is the dynamic viscosity of the fluid (Pa·s or N·s/m² or kg/(m·s))
v is the kinematic viscosity of the fluid (m²/s).

Thus, turbulent flow is meant to generally encompass Reynolds Numbers of at least 2,300, and in further configurations at least 3,500 such as for flow in a pipe, and in further configurations at least 10,000 to 800,000 such as for flow over a flat plate, and is understood to encompass Reynolds Numbers in the millions. Since the Reynolds Number depends, at least partly, on the characteristic dimension of the system, turbulent flow can also be defined as flow having irregular or chaotic eddies, and is dominated by inertial forces (rather than viscous forces) and provides a greater mixing than in laminar (non-turbulent flow).

For plants and algae, this exposes a majority of the leaves to the treated air while also increasing exposure of the leaves to the illumination source 150. The turbulent air within the turbulence zone reduces the formation of “micro-climate” zones that cause mold growth or other types of pathogens that prey on host plants, algae or fungi. It also includes a growth reaction within the plant that results in a stronger, healthier plant. For cannabis specifically, the turbulent air reduces the formation powdery mildew, gray mold, and Aspergillus that can cause health issues from inhalation or ingesting active spores from these pathogens. Moreover, spores and other debris from plants, algae and fungi 102 are directed downwardly and into return ducts 170 as set forth below.

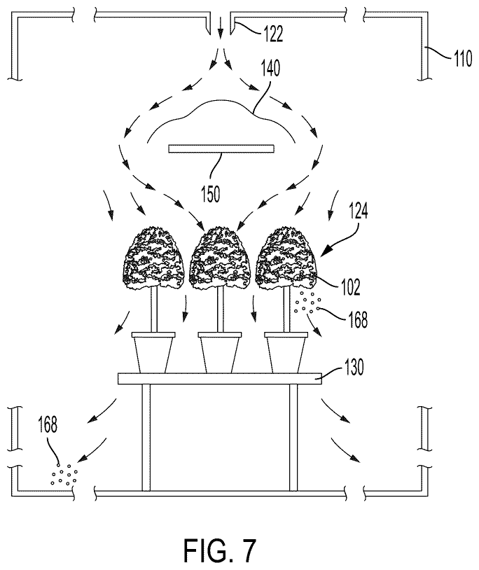

FIG. 7 shows a partial cross-section view of the example system 200 shown in FIG. 1 illustrating the center row. If a pathogen achieves replication and spore release 168, these spores will follow the air movement generally down and into the return ducts 170 for inactivation before re-entry into the room. A major source of contamination 168 in grow rooms travels in on the shoes or clothing of staff working in the space. Since these are likely knocked off near the floor the general air movement into the return ducts 170 in this region carry these spores into the system 200 for inactivation. In general, the cleanest air is provided from the system 200 directly to the plants 102. In one embodiment, the treated airflow is turbulent flow that passes through the plants 102 at a velocity that is sufficient to provide an optimal transfer of CO₂ to the plants 102. Moreover, in an embodiment, the treated airflow passes through the plants 102 at a velocity sufficient to reduce stagnant air zones within the foliage of the plant 102. That is, the turbulent flow of the treated air in one embodiment exposes a majority of the leaves of a plant within a cross-sectional volume of the plant to the treated air. Such velocity in one embodiment is in the range of between 1-3 m/s. In another embodiment, the velocity is approximately 1 m/s. The turbulent flow of air also contributes to a more even distribution of temperatures within the grow room 112, which can otherwise vary significantly due to heat generated by lighting systems, the natural tendency of warm air to rise (convention), equipment operation, and the ventilation system's efficiency.

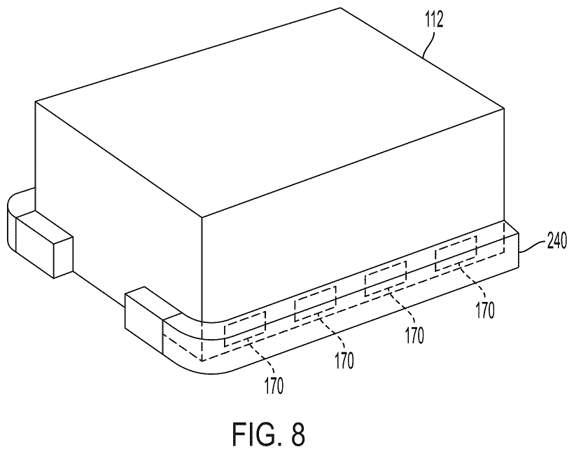

As shown in FIGS. 1 and 8, return duct vents 170 in certain embodiments are located in a linear array along each side near the floor. In an embodiment, return duct vents 170 are positioned along at least two outside walls of the grow room. As shown in FIG. 8, a series of return duct vents 170, in an embodiment, provide a linear set of return ducts 170. This creates a fluid pattern that is relatively constant at each cross section along the long axis of the grow room. Given a constant pattern, the problematic microclimate “dead zones” are reduced. It is within these dead zones that a higher probability of pathogen germination/replication occurs.

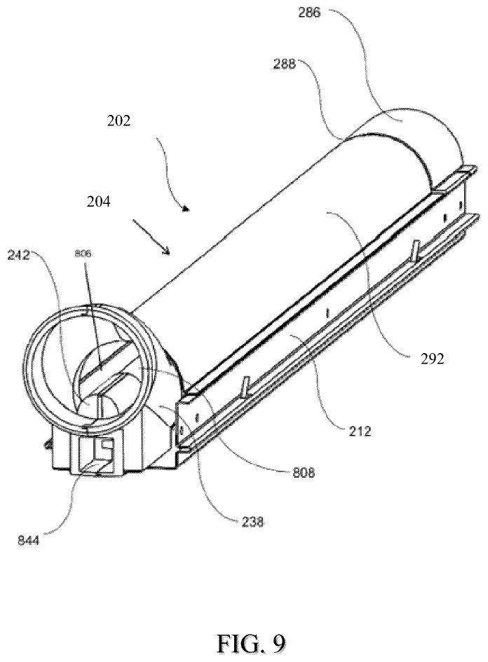

The pathogen reduction system 200 and certain components thereof are shown in FIGS. 9-12. For purposes of the present description, the illumination and pathogen reduction system 200 is set forth as having three airflow pathogen reduction ducts 202 coupled to supply ducts 120. However, it is understood, that the disclosure is not limited an illumination and pathogen reduction system 200 having three airflow pathogen reduction ducts 202, which can include one or more airflow pathogen reduction ducts 202. The illumination and pathogen reduction system 200 can be in communication with a processor as described in more detail below. Further, the illumination and pathogen reduction system 200, including at least one airflow pathogen reduction duct 202 in one embodiment is an HVAC-type or other type of heating, ventilation, and/or air-conditioning system. The term illumination and pathogen reduction system 200 can encompass any device capable of killing pathogens in an airflow and embodiments of the present invention are not limited to the particular configuration of illumination and pathogen reduction system 200 or airflow pathogen reduction ducts 202. By pathogen, it is meant any virus, bacteria, molds fungi, or other disease-causing microorganism. For example, in one embodiment, the pathogen includes microorganisms that prey on host plants 102. The emerging cannabis industry specifically has powdery mildew, gray mold, and Aspergillus potentials that can cause health issues from inhalation or ingesting active spores from these pathogens.

The illumination and pathogen reduction system 200 provides pathogen reduction in grow rooms. In an embodiment, the illumination and pathogen reduction system 200 has at least a 40% inactivation of strawberry gray mold spores caused by a fungus, Botrytis cinerea (Botrytis rot). The airflow pathogen reduction duct 202 includes an illumination chamber 292. In one exemplary embodiment, the illumination chamber 292 is a “single pass” exposure chamber having a single pass illumination channel defined by an elongated housing 204. The elongate housing 204 enables a maximum cross section and linear length for the exposure chamber and therefore less resistive force on the air flow through the pathogen reduction duct 202. The illumination chamber 292 includes an inner highly reflective layer 206 and a UV-C radiation source 208. In an exemplary embodiment, the inner layer 206 is a lining or coating having a reflectance in the range of 85%-97%, and more preferable, approximately 90%-97% and even more preferably approximately 97%. In one exemplary embodiment, the illumination chamber 292 includes a layer of anodized aluminum creating a mirror-like surface and providing approximately 90% reflectance. In another exemplary embodiment, the lining is a Teflon based diffuse reflective layer providing a reflectance of up to 97%. In yet another exemplary embodiment, the layer 206 is a specialized coating that provides a reflectance of up to 97%. Such lining can include a UV-C reflectance sheet from Porex Filtration Group that reflects 97% of UV-C light. Multi-layers of coatings or linings may further be provided. The interior of the illumination chamber 292 with a highly UV-C reflective material will increase the effective optical power or multiplier, as discussed below. The coating or lining, in one exemplary embodiment is transparent to other types of radiation, including VIS radiation, UV-A radiation and UV-B radiation. The illumination chamber 292 further includes UV-C radiation source 208 as a UV generator or UV emitter. The UV-C source 208, in one exemplary embodiment is at least one LED. In another exemplary embodiment, the UV-C source 208 is an elongated bulb or lamp arranged parallel to the flow direction of the air. For example, the UV-C, in an embodiment is a standard low-pressure mercury vapor lamp. The UV-C source 208 may alternatively include an excimer lamp or a pulse xenon lamp. For example, the Larson Electronics Far UV 222 nm, 150 W Excimer Lamp, commercially available at www.larsonelectronics.com, may be used. Such an excimer lamp utilizes Krypton Chloride (KrCl) to provide 222 nm UV-C light. In yet another exemplary embodiment, the UV-C source is a pulsed Xenon lamp. A Xenon UVC lamp provides a wide spectrum of wavelengths instead of a single wavelength. This wider spectrum of wavelengths (starting at 170 nm) provides a broader antimicrobial effect, which means they have the ability to inactivate more pathogens. An example of a Xenon UVC lamp that can be used is Ushio 5000262, UPX-44 Pulse Xenon Lamp, commercially available at www.ushio.com.

It should be appreciated that the length of the airflow pathogen reduction duct 202 can be determined by the required exposure (E=Photon density*time), where t is determined by flow rate and length. Further, it should be appreciated that the Photon density is proportional to the number of LEDs used in the system. In an embodiment, the airflow pathogen reduction duct 202 has an extrusion length of the illumination chamber 292 of approximately 50 cm and a cross-sectional area of 19.6 cm². Such airflow pathogen reduction duct 202 has, in one embodiment, approximately 8 LEDs, such that 10 airflow pathogen reduction ducts 202 include 80 LEDS which can deliver approximately 170 m³/hr (1000 ft³/min) of treated air. An array of pathogen reduction ducts 202 can be included within a duct system of an HVAC system 200. In a duct array where pressure is provided by the HVAC system the array size is based on utilizing the throughput of the airflow pathogen reduction duct 202 at the certified rate. For example, if the throughput of the HVAC system was 250 cfm and the device certification is 125 cfm then 2 airflow pathogen reduction ducts 202 are required. If the HVAC system was 1250 cfm then 10 airflow pathogen reduction ducts 202 are needed and so forth. In another embodiment, the extrusion length of the illumination chamber 292 is approximately 600 mm, the diameter of the illumination chamber 292 is approximately 116 mm. With a throughput of the HVAC system of 100 cfm, the system 200 can deliver approximately 170 m³/hr (1000 ft³/min) of treated air with 400 Watts.

In one exemplary embodiment, the illumination and pathogen reduction system 200 a sensor circuit board located internally in cavity 246 that has window 260. This sensor circuit board monitors key operational conditions, including but not limited to ambient temperature points within the assembly, UV-C power measurements within the cavity, and pressure differentials between the flow path and atmosphere. This data is communicated to control software having programming logic to ensure safety, adequate pathogen inactivation performance, and cfm throughput.

The illumination and pathogen reduction system 200 includes a pressure generator. In an embodiment, the pressure generator is an HVAC blower within the supply plenum 290. Control hardware and software having programming logic are integrated into the overall illumination and pathogen reduction system 200. The plurality of pathogen reduction ducts 202 are typically configured into an array.

Each pathogen reduction duct 202 operates at a given cfm that has been certified to decrease surviving pathogens by desired orders of magnitude. Depending on the grow room size, configuration, and desired number of air exchanges per hour (ACH), the number of reduction ducts 202 is chosen to meet the overall cfm required for the system design. For example, if the system design requires 1000 cfm at the tested rating of 150 cfm for each pathogen reduction ducts 202, then 7 pathogen reduction ducts 202 populate the array to provide a maximum 1050 cfm. The pathogen reduction ducts 202 are connected to a supply side plenum 290, which distributes the supply side air from the HVAC system evenly between elements of the array. Each reduction duct 202 delivers treated air to the grow room. In some retrofit applications the outputs could be recombined in a similar plenum as a “splice-in” to existing air distribution systems.

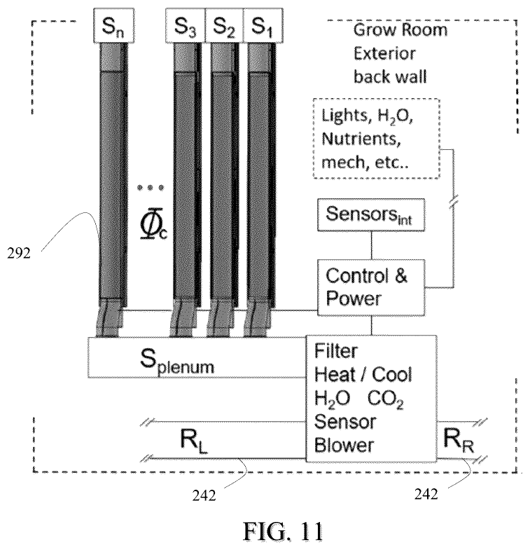

Power and control from a common computer having programing logic receive operational feedback from each reduction duct 202 in the system 200. This information is used to adjust balancing dampers 320 to ensure a desired cfm through each reduction duct 202. In an embodiment, the return ducts enter the system 200 near the floor of the grow room and act as a return plenum 240 for the series of return ducts 242 located along the left and right walls near the floor, labeled as RL and RR respectively in FIG. 11. This embodiment includes the system 200 mounted on the back wall or floor behind the grow room, a set of supply ducts 120 delivering clean air to the interior of the grow room and a set of return ducts 242 along the left and right wall near the floor. The front of the grow room in an embodiment, provides access to the grow room via a door or other means.

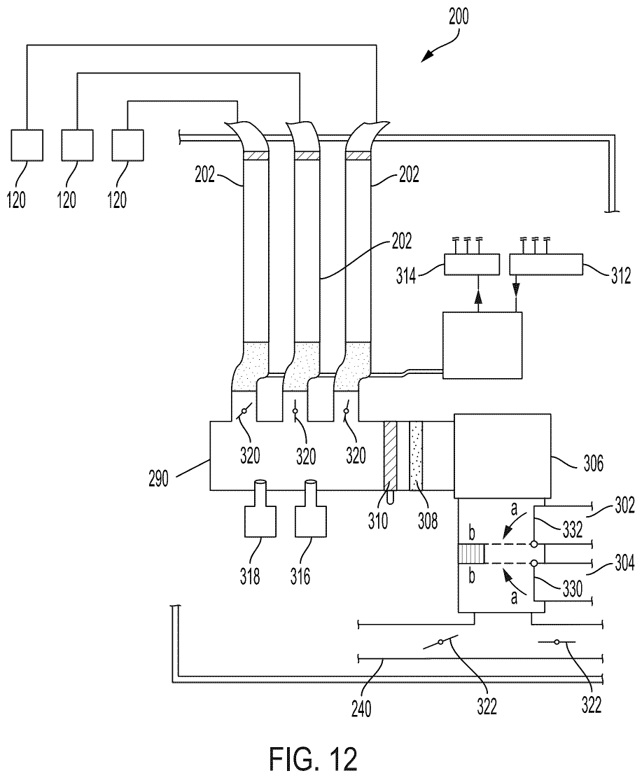

As shown in FIG. 12, in one embodiment, in an illumination and pathogen reduction system 200, an outside air supply 302 can optionally be added to the illumination and pathogen reduction system 200. That is, in one position, the bypass valves 330 and 332 are in position “a” such that the air return 240 air is directed back into the system 300. In another position, bypass valves 330 and 332 are set such that inside air is exhausted and outside supply air is added to the illumination and pathogen reduction system 200. Bypass valves 330 and 332 can be partially set to incorporate a desired percentage of outside air. The percentage of outside air may be selected according to various conditions. For example, air that is unbalanced in CO₂, O₂ and temperature can be exhausted and replaced with balanced CO₂, O₂ and lower temperature air. For example, if the temperature differential is large, then the amount of outside air incorporated into the illumination and pathogen reduction system may be large. If the temperature of the outside air is hot and the temperature differential is small, then gains from use of outside air may be small, so a low or no percentage of outside air may be selected. The percentage of outside air may also be selected according to the desired CO₂ levels within the grow room 112. Not having to supply CO₂ from tanks could provide cost saving advantages. Using natural CO₂ is lower cost than using CO₂ generators.

A blower 306 is fluidly coupled to the system 200 and exhaust supply 304 and a particular filter 308 is positioned on the supply side as shown. For example, a filter may prevent airborne particles between 0.3 and 1.0 micrometers in diameter to pass. In another embodiment a high efficiency particulate air (HEPA) filter is used. Air passing through a heat exchanger 310 would control the room interior to within desired levels. Any standard HVAC type of exchanger can be used. Interior sensors 312 coupled to a controller 314 regulate the environment of the grow room and provide data that may require a water supply 316 for humidity control and a CO₂ supply 318 to replace the CO₂ consumed in the photosynthesis process. Balancing dampers 320 ensure that air flow is configured to desired levels through each pathogen reduction duct 202 and each side of the room. This aids in establishing the interior flow patterns within the vegetative canopy throughout the growing volume. Balancing dampers 322 ensure that airflow is configured to desired levels through the blower 306. For example, return airflow through the return duct line fluidly connected to the return flow control box is configured to balance the airflow removed from the right and the left of the grow room 112.

It should be appreciated that the present disclosed airflow reduction system 200 provides the following features:
1) The population percentage in the π* metastable state in relation to the corresponding π rest state at any given time that pathogens are within the illumination chamber,
2) The UV-C power to wall efficiency that is greater than one due to the effective “reuse” of generated photons passing through the air to be sanitized multiple times. Using a conservative estimate of a factor of 5, The effective UV-C power in watts exceeds the electrical power required to generate the source UV-C illumination and,
3) The chamber geometry that details a very high percentage (>90%) of the possible 4π steradian attack angles to pathogens simultaneously for much of the illumination chamber length.

In laser operation, it is well known that the excited state population must exceed the rest state for achieving laser action. This situation is called a population inversion where a large enough percentage of excited state exists that can be “stimulated” by previously generated photons within the amplification cavity. To reach this conversion the material must be “pumped” through electron bombardment, electric field oscillations or other means.

Using a combination of containment of generated photons, a proximity of pathogens to the source and high number of attack angles provided by the chamber design, a population inversion of excited bases may occur. Given the reaction to create dimers for the prevention of pathogen replication is only possible for 1 millisecond, it requires the extreme photon density achieved with this design as well as optimization of the number of pathogen attack angles the highest probability of nucleoid base absorption, dimer formation, and subsequent inactivation by the disruption of the transcription process.

In laser analogy, the population of targeted nucleoid bases is pumped by photon bombardment to an excited state thereby creating a population inversion which enables adequate dimer formation or other damage such that the photoprotection and DNA repair mechanisms are overwhelmed preventing replication.

Further the LED grow illumination can be tailored to reduce photo repair by eliminating the sub 400 nm light present in sunlight. The sub 400 light also stimulates conidia germination and light greater than 400 inhibits germination. Photons below 400 have sufficient energy to be absorbed into formed dimers which then enables the dimer to split, and DNA repaired. Photons above 400 do not have the energy required. 400 is not a hard breakpoint but does represent where a transition from constructive to destructive effects occur.

A last characteristic is that it is often stated that Melanin layers present in many conidia protect the core by absorbing UV-C light. While true, the melanin layers are thin and thus the amount of light that gets through is still sufficient to inactivate and overwhelm repairs mechanisms. Absorption coefficients increase dramatically as the wavelengths get smaller throughout the UV-C range. While no current data could be found for wavelengths below 254, the projection indicates that lower wavelengths such as 222 nm would have at least an order on magnitude less transmission.

### Exemplary Embodiment of a Standard Grow Room

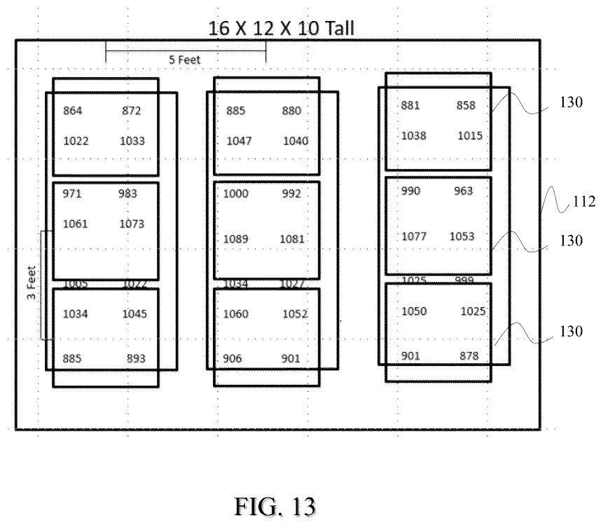

An exemplary grow room 112 is shown in FIG. 13. In one exemplary embodiment, grow room 112 is 16 feet by 12 feet with a height of 10 feet and includes an airflow system 200 having a UV-C source for delivering treated air to predetermined areas. The grow room 112 may include three sets of three tables 130, wherein each table 130 is approximately 3 feet by 3 feet. While FIG. 13 shows three tables each having three general areas for objects requiring illumination, it should be appreciated that any base having a support surface for receiving an object can be used. In one exemplary embodiment, the objects are plants 102. In another exemplary embodiment, the objects are fungi. In some settings, a powdery mildew, gray mold and/or Aspergillus forms on the objects, which can cause health issues from inhalation or ingesting active spores from these pathogens.

The numbers presented on each table 130 are each a measure of light in terms of PPFD units. PPFD units are photosynthetic photon flux density which corresponds to the number of micro moles radiated by the light source per square meter per second, expressed as mirmormol/m2s. Photons are light particles. Thus, PPFD measures the light (photons) that reach the target per second. It should be appreciated that natural or artificial light can be used to support photosynthesis of a plant. LED lamps are an example of artificial light that is used to support photosynthesis of a plant. Plants convert light energy into chemical energy through photosynthesis. Photosynthetically active radiation, or PAR, is used to refer to the light output of LED lamps. PAR defines the type of light needed to support photosynthesis in plant life. It should be appreciated that the growth rate of an organism is directly related to the light irradiation. Ultraviolet light, having a wavelength range between about 300 and about 400 nm and near-infrared light (far-red light), having a wavelength range between about 700 and about 800 nm will affect the biochemical reaction and appearance of organisms. The light in the spectrum range of between about 400 nm and 700 nm is called PAR and closely related to the photosynthesis of plants and algae. The total light (photons) emitted per second by a light source with a wavelength of between about 400 nm and 700 nm is the photosynthetic photon flux (PPF) and can be measured by the number of micro moles of photons emitted by an artificial light source per second micromol/s. The unit of photosynthetic irradiance does not reflect the influence of wavelength. PPFD is a physical quantity related to the radiation distance, which is inversely proportional to the square of the radiation distance. 1 ppfd is the number of photons per second radiating from a surface of 1 square meter. Photosynthetic photon flux density PPFD is the energy absorbed by plants for photosynthesis. The flux density of PPFD is a surface measurement so should be called “surface density” instead. This should not be confused with our definition of flux (Photon) density for a volume element. Thus, in one exemplary embodiment, the grow room 112 includes illumination sources 150 which are LED lamps having a spectrum range of between about 400 nm and 700 nm to provide photosynthetically active radiation in an illumination zone. The objects are placed in the illumination zone to be exposed to the photosynthetically active radiation. By illumination zone, it is meant a volume of space wherein illumination is desired, for example, a volume of space having the canopy of a plant or plants.

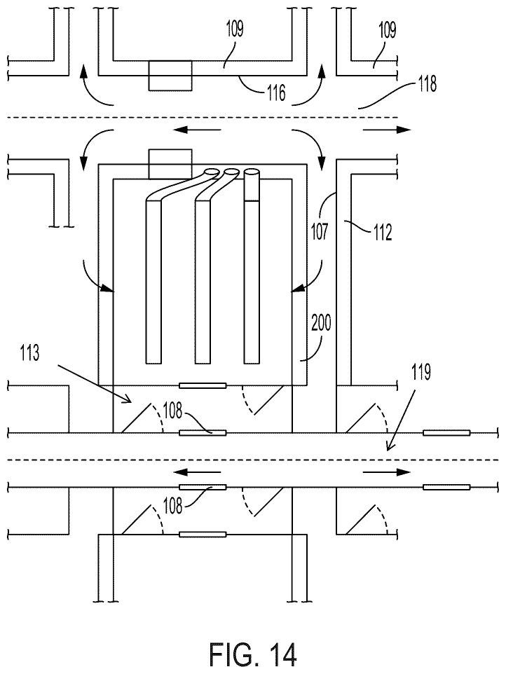

FIG. 14 details a top view of a grid application of a plurality of grow rooms 112. All back walls 116 would line either side of a maintenance hallway 118. Each side of one of the grow rooms 112 is accessible from the maintenance hall 118. The front of each room lines a clean worker hallway 119 isolated from the environments in each room and controlled to reduce contamination from the outside. In one configuration, a central area 114 includes a portion of the pathogen reduction system 200, including a substantial portion of the airflow pathogen reduction ducts 202. Observation windows 108 are shown as well as entry air locks 113 for worker prep for room entry.

### Exemplary Embodiment of a Shipping Container Grow Room

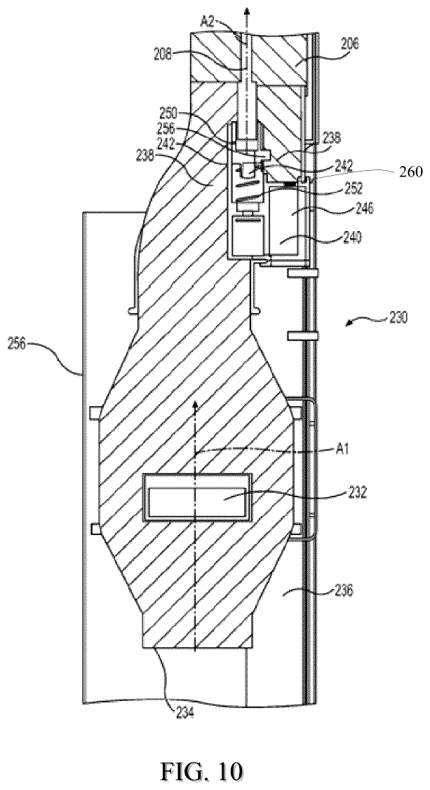

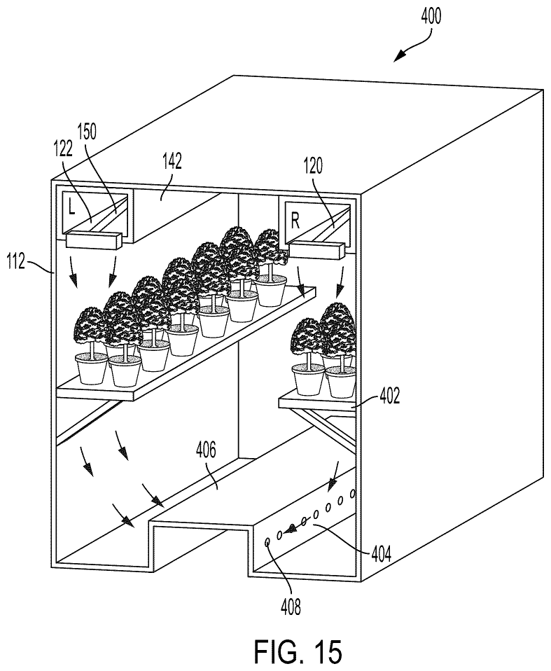

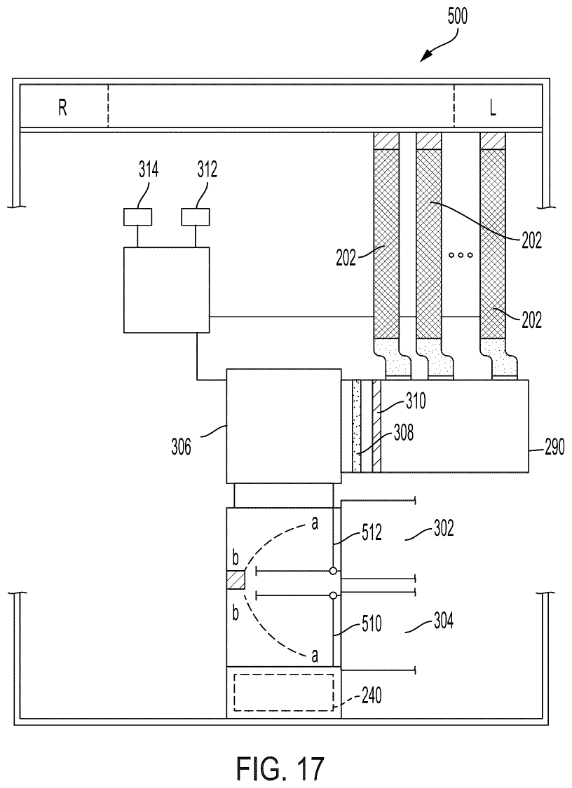

As shown in FIGS. 15-17, in an embodiment, a grow room 112 may be built within an enclosure 400, such as a shipping container. Exemplary embodiments of a shipping container measure approximately 8 feet in width by 9.5 feet in height by 40 feet in length. Finished grow rooms 112, having an airflow system 200 (see FIG. 12) or 500 (see FIG. 17) having a first flow path and a UV-C source 208 (shown in FIG. 10) optically coupled to the first flow path for treating a volume of air, at least one turbulence generator 140 coupled with a lighting housing 142, at least one base 402 having an adjustable height, and a return duct, can be shipped to end locations. The finished grow rooms 112 may include plants or fungi 102 that have reached a desired maturity or are in the process of growing.

In certain exemplary embodiments, it is desirable to not exceed the 8′×9.5′ profile on the outside of the enclosure, such as a shipping container. As such, components are typically located on the “inside” of that profile. FIG. 15 shows an illumination source 150, such as an integrated LED, and treated air supply ducts 120 along the upper left and right side fixed to the ceiling (top wall) as shown.

In an exemplary embodiment, the enclosure 400 includes an internal return duct 404 that also provides an elevated walkway 406 between the bases 402, such as the plant tables. These tables, in one exemplary embodiment, adjust vertically or along a y-axis to keep the foliage canopy of the plants (or fungi) 102 at the desired distance from the illumination source 150, such as a LED or plurality of LEDs, which is also the desired distance to ensure turbulence thought the canopy presented in the turbulent zone 124 caused by the treated air supply venting in combination with the turbulence generator 140.

The floor mounted return duct 406 is structural to support the weight of workers and has apertures or ports 408 along its length to ensure a consistent return flow along the length of the room 112 through the return duct line 406. For example, the floor mounted return duct may be capable of bearing weight of at least 250 lbs. The apertures 408 may be adjustable to create a proper flow distribution. In one exemplary embodiment, the apertures 408 along the return duct 406 are smaller on one end than the other to create a proper flow distribution. For example, the apertures 408 positioned farthest away from the blower of the airflow system 200 (or 500 as shown in FIG. 17) may be larger than the apertures 408 positioned closest to the blower of the airflow system 200 or 500. For similar reason, the supply “linear” vents 122 of the supply ducts 120 may change in size along the length, wherein the vents 122 farther away from the blower of the airflow system 200 or 500 are larger than the vents 122 positioned closest to the blower of the airflow system 200 or 500. In one exemplary embodiment, the apertures 408 and vents 122 are graduated in size, with the smallest apertures 408 and vents 122 located closest to the airflow system 200 or 500. This accounts for the lower pressure differential as the distance from the blower of the airflow system 200 or 500 increases.

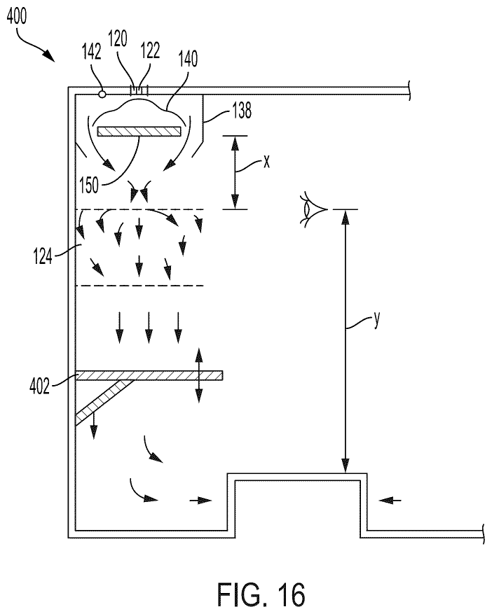

FIG. 16 shows the left side of a symmetrical flow condition for each “slice” along the length of the grow tables 130. In this exemplary embodiment, treated air supplied by the ceiling fixed mounted integrated LED supply duct 120 is directed to cause a turbulent zone 124 at the desired location of an object as determined by optimal illumination by the illumination source 150. For example, the object may be at least one plant 102 having a canopy of leaves. The canopy may be disposed in the illumination/turbulent zone 124 to be exposed to the turbulent treated airflow. That is, in one embodiment, the turbulent zone 124 encompasses foliage of a plurality of plants. The downward average air velocity passes through the canopy around photosynthetically active radiation. That is, by “illumination zone,” it is meant a volume of space where illumination is desired, for example, a volume of space having the canopy of a plant or plants. The location of the turbulent zone and illumination zone is the same or overlapping. The turbulent air ensures that the desired velocity of air goes across the leaves greatly reducing the probability of “micro-climate” zones that may inspire mold growth, the “trunks” and soil pots, through perforated tables and is drawn into the return “walkway” duct. Since the illumination source 150 is fixed mounted within the supply ducts 120, the tables 402 adjust vertically to position the canopy at a desired height throughout the growing cycle.

The height of the walkway 406 is determined to provide the optimal visual angle for workers (dimension “y”). It may be desired for the table 402 to pull into the isle enabling a worker access to the outer rows. A walking shelf along the outer side would provide a similar height. Table height adjustment could be along the wall as indicated in FIG. 16, from underneath, or from above with tables suspended.

Since each slice along the linear length has an equivalent air path an optimal distribution of treated air and turbulence is provided along the length.

FIG. 17 shows the airflow system 500 on the outside end of the container 400. An array of pathogen reduction ducts 202 is used to achieve the level of pathogen reduction desired for a given overall HVAC system design.

In one exemplary embodiment, the system for a container having approximately 3000 cubic feet, includes two pathogen reduction ducts 202, each having an airflow volume of approximately 150 cfm to provide 6 ACH. For example, an 8′×9.5′×40′ container has 3040 cubic feet of air.

If an air exchange of 6 air changes per hour (ACH) is desired, two pathogen reduction ducts 202 can be provided, each running 152 cfm. 12 ACH would require 4 devices, 18 ACH would require 6 devices, etc.

The illumination and pathogen reduction system 500 includes a pressure generator. In an embodiment, the pressure generator is an HVAC blower attached to the supply plenum 290. Control hardware and software having programming logic are integrated into the overall illumination and pathogen reduction system 500. The plurality of pathogen reduction ducts 202 are typically configured into an array.

Each pathogen reduction duct 202 operates at a given cfm that has been certified to decrease surviving pathogens by desired orders of magnitude. Depending on the grow room size, configuration, and desired number of air exchanges per hour (ACH), the number of reduction ducts 202 is chosen to meet the overall cfm required for the system design as described above.

Power and control from a common computer having programing logic receive operational feedback from each reduction duct 202 in the system 200. This information is used to adjust balancing dampers 320 to ensure a desired cfm through each reduction duct 202. In an embodiment, the return ducts 404 (see FIG. 15) enter the system 200 near the floor of the grow room 112 and act as a return plenum 240

As shown in FIG. 17, in one embodiment, in an illumination and pathogen reduction system 500, an outside air supply 302 is not mixed. In this embodiment, a bypass value is used such that the air return from return 240 is directed back into the system 500. That is, bypass valve 510 and bypass valve 512 are in position “a” to block outside air from coming in. This permits air in the container 400 to be recycled and be treated. Alternatively, bypass valve 510 and bypass valve 512 can be in position “b”, wherein outside air is added to the system and air returned from return 240 is exhausted out of the system 500. It should be appreciated that bypass valves 510 and 512 can be partially set to incorporate a desired percentage of outside air.

A blower 306 is fluidly coupled to the outside air supply 302 and exhaust supply 304 and a particular filter 308 is positioned on the supply side as shown. For example, a filter 308 may prevent airborne particles between 0.3 and 1.0 micrometers in diameter to pass. In another embodiment a high efficiency particulate air (HEPA) filter is used. Air passing through a heat exchanger 310 would control the room interior to within desired levels. Any standard HVAC type of exchanger can be used. Interior sensors 312 coupled to a controller 314 regulate the environment of the grow room and provide data that may require a water supply for humidity control and a CO₂ supply to replace the CO₂ consumed in the photosynthesis process.

It should be appreciated that plants and fungi will have different temperature needs and these can be accommodated within the illumination and pathogen reduction system 200. As an example, strawberry plants have particular temperature requirements, one of which is a gradual increase in temperature at the start of the season. This mimics the transition from winter to summer. This helps the plants grow larger, so they can produce and manage more fruit later. Strawberry grow room temperatures therefore typically begin around 8 °C (46 °F) at night and 16 °C (60 °F) during the day. Over several weeks the temperature is gradually increased, reaching 10-12 °C (50-54 °F) at night, and 20-24 °C (68-75 °F) during the day. When strawberry plants flower, it's best to maintain a temperature range of 16-20 °C (60-68 °F). However, once the plants bear fruit, a range of 15-16 °C is ideal for maturation. Ideal temperatures differ not only for different development stages, but also between strawberry varieties. They also depend on other environmental conditions, such as humidity, lighting, etc.

### Exemplary Embodiment of a Vertical Grow Room

In certain embodiments, the plant, fungi or algae 102 is located below the illumination source 150. In other embodiments, vertical rows of plants, fungi or algae 102 are provided in a vertical farming CEA system. The vertical farming CEA system typically includes specialized illumination sources 150 to provide optimal light distribution across densely stacked plant growth layers. For example, inter- or intra-canopy lighting can be used as an illumination source 150. In this instance, the lighting is positioned within the plant canopy or between plant shelves. This embodiment is especially useful for crops with thicker canopies or taller growth habits, such as certain leafy greens or herbs. Typically, in this embodiment, LED strips or bars are vertically mounted along the sides of the growing shelves or trays. These strips run the length of the shelves, illuminating plants from the side. Overhead LED panels or bars can still be used in conjunction to provide top-down illumination. An alternative type of illumination source 150 is layered or shelf-style lighting. In this instance, each layer or shelf of plants 102 has a set of lights positioned next to it. Typically, in this embodiment, a multi-tier racking system is used, where each tier or shelf supports a flatbed of plants. Directly above each bed, LED panels or bars are installed. These lights are usually adjustable in height, allowing for flexibility based on plant growth stage or type. It should be appreciated that the illumination source 150 should be suitable for the crop's needs, considering factors like light spectrum, intensity, and photoperiod. Additionally, thermal management is important in vertical farming setups because of the proximity of lights to plants and the potential for heat buildup.

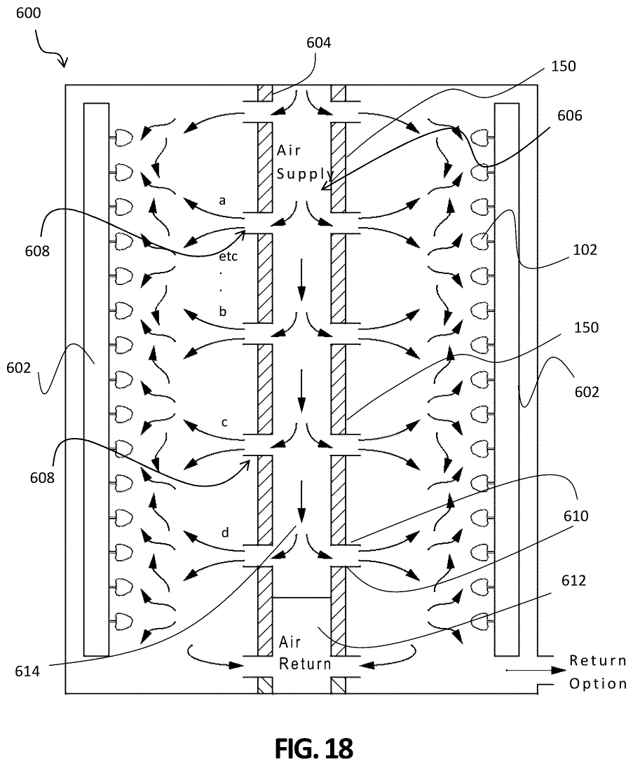

As shown in FIG. 18, an exemplary embodiment of a vertical farming CEA system 600 is provided. In this system 600, plants 102 are arranged on vertical panels or walls 602, facing the illumination source 150. The illumination source 150 is mounted on the opposite wall or panel 604. This set up provides a “corridor” of light between the plant wall 602 and the light wall 604. Thus, plants 102 receive a uniform light distribution from the illumination source 150. The vertical farming CEA system 600 further includes a duct 606 (or ducts) projecting a treated air supply 614 toward the plants 102. The duct 606 is spaced from the plant wall 602 and includes a plurality of orifices 608. In one embodiment, each orifice 608 includes a turbulence generator 610 within or along the perimeter of each orifice 608. The treated air supply duct 606 and turbulence generator 610 directs a turbulent airflow toward the plants 102. In one embodiment, the plurality of orifices 608 are spaced along the duct 606 and vary in size. For example, each orifice 608 may be increasing larger than the previous orifice 608 as the orifices 608 are closer to the floor. In one configuration, each turbulence generator 610 is an airfoil that discharges air through the foliage of the plants 102. Depending on the location of the airfoil, the air may be directed up, down, straight, or a combination of these. Thus, turbulent zones are created through the plants 102. The system 600 includes a return duct 612 proximate the floor. The vertical farming CEA arrangement can provide very uniform light distribution, reducing the possibility of shadowed or light-starved plants. It also allows easy access to plants for monitoring, harvesting, and maintenance since there's no overhead lighting obstructing the grower. It can be aesthetically pleasing and is sometimes used in commercial or public spaces like restaurants or atriums where the design is as important as production.

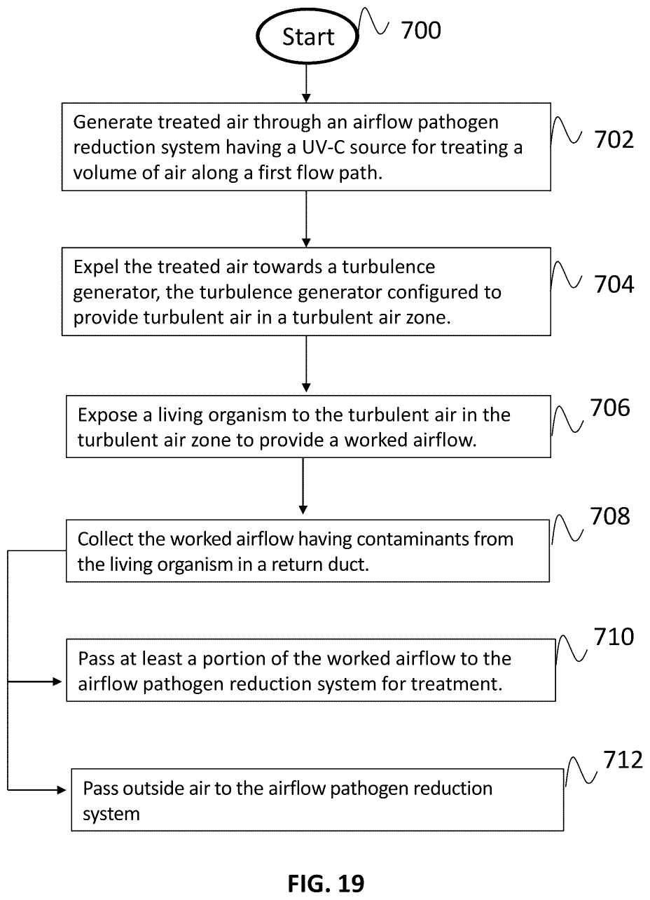

FIG. 19 presents a summary of the above teachings for treating a living organism in a controlled environment agricultural system in the form of a logic diagram. The logic diagram of FIG. 19 may be considered to illustrate the operation of the method 700. Block 702 presents generating treated air through an airflow pathogen reduction system having a UV-C source for treating a volume of air along a first flow path. Then block 704 presents expelling the treated air towards a turbulence generator, the turbulence generator configured to provide turbulent air in a turbulent air zone. According to block 706, a living organism can be exposed to the turbulent air in the turbulent air zone to provide a worked airflow. Next, block 708 presents collecting the worked airflow having contaminates from the living organism in a return duct. In one embodiment, as shown in block 710, at least a portion of the worked airflow is passed to the airflow pathogen reduction system for treatment. In the same or alternative embodiment, outside air is passed to the airflow pathogen reduction system according to block 712.

It is projected that energy usage has become and will become a bigger factor as market production volumes increase. Lower prices mean lower profits that can be recovered in part by reducing energy usage. Moving from high intensity discharge lamps to LEDs is an energy cost savings. By delivering the turbulent flow directly at the canopy a lower ACH can be used.

Lowering the ACH reduces energy requirements for the HVAC system. Current systems rely on pre-operation chemical sanitization to reduce the probabilities of plant pathogen growth. Then once plants are added a “sealed” air operation is used to not allow pathogen introduction from the outside environment. This requires that the HVAC must provide the cooling and heating necessary to provide desired growing conditions. In addition, CO₂ and O₂ must be monitored and adjusted. All these requirements relate to growing overhead costs.

Since the disclosed system provides sanitization of air on the supply side of the room, air can be brought in from the outside and exhausted during the active grow. Now when cooling is needed it can be accomplished by the introduction of outside air if lower than inside and provide heat if the outside is higher than desired. In either case the air is sanitized so pathogens are controlled. Outside air will also balance the CO₂— O₂ concentrations naturally such that the demand on those internal systems is reduced.

In all, the system will use less energy than alternative means and relate to higher profits for the grower. Lowering the use of chemicals also is desirable for more “organic” production.

While the invention has been described in connection with certain presently preferred aspects thereof, those skilled in the art will recognize that many modifications and changes may be made therein without departing from the true spirit and scope of the invention which accordingly is intended to be defined solely by the attended claims.

1. An illumination and pathogen reduction system for controlled environment agriculture comprising:
an airflow pathogen reduction system having a first flow path and a UV-C source optically coupled to the first flow path for treating a volume of air, the volume of treated air expelled along a second flow path;
an illumination source having photosynthetically active radiation directed towards an illumination field; and
a turbulence generator within the second flow path, wherein the turbulence generator provides a turbulent flow of treated air towards a desired location.

2. The illumination and pathogen reduction system of claim 1, wherein the desired location is a turbulent zone including at least one living organism.

3. The illumination and pathogen reduction system of claim 2, wherein the turbulent zone encompasses foliage of a plurality of plants.

4. The illumination and pathogen reduction system of claim 1, wherein the illumination source is a plurality of LEDs configured to illuminate the illumination field.

5. The illumination and pathogen reduction system of claim 1, wherein the turbulence generator is a first air deflector, the first air deflector comprising a deflector body within the second flow path.

6. The illumination and pathogen reduction system of claim 5, wherein the deflector body further comprises an undulating outer surface with a leading portion and trailing edges, wherein the turbulent zone is located within the second flow path following the trailing edges.

7. The illumination and pathogen reduction system of claim 1, wherein the turbulence generator is an airfoil configured to provide the turbulent flow of treated air towards the desired location.

8. The illumination and pathogen reduction system of claim 2, wherein the illumination source is within a fixture, and wherein the fixture is moveable along at least one axis to locate the illumination source at a selected distance from the at least one living organism.

9. The illumination and pathogen reduction system of claim 2, wherein the at least one living organism is positioned on an adjustable surface to locate the at least one living organism a selected distance from the illumination source.

10. The illumination and pathogen reduction system of claim 3, wherein the airflow system comprises:
an HVAC system having a controller, an air handling unit, a supply duct line, and a return duct line;
a return flow control box fluidly connected to the return duct line of the HVAC system; and
at least one airflow pathogen reduction system, the airflow pathogen reduction system having an airstream intake for receiving a volume of untreated air located at a first position, an airstream duct for moving a volume of treated air to a room, an input power source operably connected to the UV-C source, an illumination channel along the first flow path having an inner highly UV-C reflective surface providing an optical effective power that exceeds the input power to the UV-C source, wherein the air handling unit of the HVAC system is fluidly connected to the airstream intake, the air handling unit configured to impart a flow from the airstream intake to the airstream duct, and an airflow control system fluidly connected to the airstream duct.

11. The illumination and pathogen reduction system of claim 2, wherein the turbulent flow of treated air is passed through the at least one living organism providing worked airflow, and wherein at least a portion of the worked airflow is received within the return duct line to be treated by the airflow pathogen reduction system.

12. The illumination and pathogen reduction system of claim 1, wherein a portion of outside air is mixed with the worked airflow to be treated by the airflow pathogen reduction system.

13. The illumination and pathogen reduction system of claim 2, wherein the turbulent flow of treated air has a sufficient velocity to provide at least one of (i) an optimized transfer of CO₂ to the at least one living organism, (ii) a reduced number of stagnant air zones, (iii) a reduced temperature gradient; and (iii) an increased surface exposure area of the at least one living organism to the illumination source.

14. The illumination and pathogen reduction system of claim 1, wherein the illumination and pathogen reduction system is integrated into a shipping container, and wherein the airflow pathogen reduction system is located on the outside of the shipping container.

15. The illumination and pathogen reduction system of claim 1, where the turbulent flow of treated air has a velocity ranging between approximately 1 m/s to 3 m/s.

16. The illumination and pathogen reduction system of claim 1, wherein the illumination source has a selectable range of wavelengths.

17. The illumination and pathogen reduction system of claim 9, wherein the at least one living organism is a plurality of plants arranged in a vertical array, wherein a supply duct line is vertically spaced from the plurality plants, wherein the supply duct line includes openings for expelling the volume of treated air along the second flow path, and wherein each opening includes turbulence generators configured to provide a turbulent flow of treated air towards the plurality of plants.

18. The illumination and pathogen reduction system of claim 9, wherein the supply duct line includes vents having tapered openings, wherein the openings narrow as the distance from the airflow pathogen reduction system decreases.

19. A method of treating a living organism in a controlled environment agricultural system comprising:
a. generating treated air through an airflow pathogen reduction system having a UV-C source for treating a volume of air along a first flow path;
b. expelling the treated air towards a turbulence generator, the turbulence generator configured to provide turbulent air in a turbulent air zone;
c. exposing a living organism to the turbulent air in the turbulent air zone to provide a worked airflow; and
d. collecting the worked airflow having contaminants from the living organism in a return duct.

20. The method of claim 18, further comprising passing at least a portion of the worked airflow to the airflow pathogen reduction system for treatment.

21. The method of claim 18, further comprising the step of adjusting the distance between the living organism and at least one of the illumination source and the turbulence generator.

22. The method of claim 18, further comprising passing outside air to the airflow pathogen reduction system.

23. An indoor grow room enclosure comprising:
an airflow pathogen reduction system having at least one illumination chamber subassembly comprising first flow path and a UV-C source optically coupled to the first flow path for treating a volume of air, the volume of treated air expelled along a second flow path;
a turbulence generator within the second flow path, the turbulence generator providing turbulent airflow in a turbulent zone of treated air;
an illumination source directed towards an illumination zone, the illumination source capable of illuminating the illumination zone;
a first base having a support surface for receiving an object requiring illumination, the base having an adjustable height for positioning the object within the illumination zone and the turbulent zone of treated air, wherein the turbulent airflow is passed through the object providing worked airflow having contaminants; and
a return duct for removing the worked airflow from an indoor grow room enclosure.

23. The indoor grow room enclosure of claim 22, wherein the indoor grow room enclosure is a shipping container having an exterior and an interior, the interior formed by sidewalls, a top wall and a bottom wall, wherein the first base is proximate one of the sidewalls and wherein a second base is proximate another one of the sidewalls opposite the first base.

24. The indoor grow room enclosure of claim 23, wherein the return duct forms a walkway within the shipping container between the first base and the second base.

25. The indoor grow room enclosure of claim 23, wherein the return duct is approximately parallel to the sidewalls and includes a plurality of apertures, each aperture increasing in size as the distance of the aperture from the airflow pathogen reduction system increases.

26. The indoor grow room enclosure of claim 22, wherein the object requiring illumination is at least one plant, algae or fungi.

27. The indoor grow room of claim 22, wherein at least a portion of the volume of air treated by the airflow pathogen reduction system is from outside of the enclosure.

28. The indoor grow room of claim 22, wherein at least a portion of the worked airflow is passed to the airflow pathogen reduction system for treatment.

29. The indoor grow room of claim 22, wherein the object requiring illumination is a plant having a foliage canopy, and wherein the foliage canopy is positioned within the turbulent zone of treated air to provide movement of the foliage canopy.

30. The indoor grow room of claim 22, wherein the turbulent airflow in the turbulent zone includes treated air having a velocity of between 1 to 3 m/s.
